(19) United States
(12) Patent Application Publication
(10) Pub. No.: US 2024/0342227 A1
(43) Pub. Date: Oct. 17, 2024

(54) COMBINATION OF E. FAECALIS AND AN ANTI-INFLAMMATORY AGENT AND USES THEREOF IN THE PREVENTION AND/OR TREATMENT OF RESPIRATORY DISEASES

(71) Applicants: INSTITUT NATIONAL DE RECHERCHE POUR L'AGRICULTURE, L'ALIMENTATION ET L'ENVIRONNEMENT, PARIS (FR); INSTITUT NATIONAL DES SCIENCES ET INDUSTRIES DU VIVANT ET DE L'ENVIRONNEMENT, Palaiseau (FR); UNIVERSITE PARIS-SACLAY, Gif-sur-Yvette (FR)

(72) Inventors: Muriel THOMAS, IGNY (FR); Pascale SERROR, JOUY EN JOSAS (FR); Alexandre DAVID-HACHETTE, SAINTE MAXIME (FR); Jennifer PALOMO, GUYANCOURT (FR)

(21) Appl. No.: 18/701,057

(22) PCT Filed: Oct. 13, 2022
(86) PCT No.: PCT/EP2022/078576

§ 371 Date: Apr. 12, 2024

(30) Foreign Application Priority Data
Oct. 15, 2021 (FR) FR2110973

(51) Int. Cl. A61K 35/744; A61K 31/58; A61P 29/00

(52) U.S. Cl. CPC A61K 35/744; A61K 31/58; A61P 29/00

(57) ABSTRACT

The present invention relates to the association of at least one bacterial strain of the species Enterococcus faecalis and an anti-inflammatory agent, a composition comprising same, and the uses thereof as a medicament, in particular for preventing and/or treating respiratory diseases.

### TECHNICAL FIELD

The invention relates to the combination of at least one bacterial strain of the species Enterococcus faecalis and an anti-inflammatory agent, a composition comprising them and uses thereof as a medicament, in particular for preventing and/or treating respiratory diseases.

### PRIOR ART

Chronic respiratory inflammatory diseases such as asthma, chronic obstructive pulmonary disease (COPD), allergic rhinitis, rheumatoid arthritis, but also chronic inflammatory bowel diseases (IBD) affect millions of people worldwide. These chronic diseases have a major impact on the quality of life of patients for whom no curative treatment is available.

The prevalence of these diseases greatly increases in Western countries, which constitutes a major medical, social and economic problem. Current treatments are essentially symptomatic and aim to treat or optionally prevent seizures that occur from time to time, making it possible to improve the quality of life of patients.

These treatments are notably based on broad-spectrum immunity modulators, such as corticosteroids. These are certainly very effective as one-off treatments, but may lead to harmful side effects when taken regularly. For example, inhaled corticosteroids can cause delayed growth in children, suppression of the hypothalamic-pituitary-adrenal axis and increased risk of osteoporosis. In addition, certain patients, who are refractory to corticosteroids, receive high doses causing severe side effects such as metabolic disorders or an increased risk of cardiovascular diseases (De ludicibus et al. Molecular mechanism of glucocorticoid resistance in inflammatory bowel disease. World J Gastroenterol 2011. 17:1095-108; Ora et al. Advances with glucocorticoids in the treatment of asthma: state of the art. Expert Opin Pharmacother 2020. 21:2305-2316).

Whereas congenital resistance to corticosteroids is rare, acquired resistance is more common. Congenital resistance results from mutations in the glucocorticoid receptor gene, whereas acquired resistance is multi-factorial. Regardless of the type, glucocorticoid resistance is a serious therapeutic problem limiting the response in patients with chronic inflammatory diseases. Acquired resistance can be attributed to modifications of the cellular microenvironment, which is a consequence of chronic inflammation. Multiple factors have thus been identified, in particular signaling alterations downstream of cytokine action, oxidative stress, hypoxia and serum-derived factors.

Research has also been conducted on the microbiota. A specific strain of E. faecalis was thus discovered and isolated for its anti-inflammatory properties as disclosed in patent application FR 1650656. It has particularly advantageous properties in the treatment and/or prevention of respiratory diseases such as asthma.

There is therefore a serious medical need for novel therapeutic solutions making it possible to increase the response of the tissues to glucocorticoids and thus reduce the treatment doses of the anti-inflammatory agents needed and/or increase their efficacy.

An objective of the present invention is therefore to provide a solution that is simple, effective and economical in order to reduce the anti-inflammatory treatment doses needed and/or to increase their efficacy and thus prevent and/or treat respiratory diseases.

### SUMMARY OF THE INVENTION

In order to meet this objective, the inventors have identified that the combination of an active ingredient based on bacteria of the species Enterococcus faecalis (hereinafter E. faecalis) and another active ingredient based on anti-inflammatory agents, preferentially glucocorticoids, induces a synergistic effect making it possible to reduce the doses of the useful glucocorticoid administered to a patient, including patients refractory to glucocorticoids.

The inventors thus observed that the strains alone at MOI 10 (that is, 10 cells of E. faecalis per 1 eukaryotic cell) or low-dose budesonide (0.1 nM) did not induce an effect, in vitro, on interleukin-8 (IL-8) release induced by Tumor Necrosis Factor (TNF)-α on human lung BEAS-2B cells.

However, during the co-incubation of the same dose of budesonide (0.1 nM) in combination with the strains of E. faecalis (MOI 10), the inventors observed a synergistic effect corresponding to the release of IL-8 being inhibited by 32%, whereas the inhibition of IL-8 release obtained with a 5-times higher dose of budesonide only (that is, 0.5 nM) was 55%. Thus, in order to obtain a similar effect, it is necessary to use extremely high doses of budesonide which also results in major side effects.

The study of the potentiating effect of E. faecalis on a more complex model of lung explants of axenic mice in ex vivo culture made it possible to confirm the in vitro results by observing a greater anti-inflammatory effect for the combination according to the invention (0.1 nM budesonide+E. faecalis) compared to the anti-inflammatory effect obtained with a 50-times higher dose of budesonide alone (5 nM).

The combination of a glucocorticoid and E. faecalis bacteria is thus of particular interest to reduce the useful concentration of glucocorticoids used as first-line treatment. The invention is therefore particularly suitable for patients suffering from chronic inflammatory diseases and preferentially resistant to corticosteroids.

Thus, the invention relates to the combination of two active ingredients, that is, at least one bacterial strain of the species E. faecalis (i) and an anti-inflammatory agent (ii) for use as a medicament in humans or animals.

The invention also relates to the culture supernatant obtained from one of the bacterial strains of E. faecalis and combined with an anti-inflammatory agent.

Finally, the invention relates particularly to a composition comprising the combination of at least one bacterial strain of the species E. faecalis (and/or the supernatant thereof) (i) and an anti-inflammatory agent (ii) and at least one acceptable excipient (iii). Said acceptable excipient preferentially being a pharmaceutically acceptable excipient when it is a product intended to be used as a medicament for human use or veterinary use. The composition is then a pharmaceutical composition comprising a strain of E. faecalis and an anti-inflammatory agent and at least one pharmaceutically acceptable excipient.

The invention is thus particularly suitable for using said combination or said composition according to the invention as a medicament. Indeed, the present invention is useful in the prevention and/or treatment of inflammatory diseases, in particular respiratory diseases, more particularly chronic diseases such as allergy, asthma, chronic obstructive pulmonary disease (COPD), or allergic rhinitis.

Other chronic inflammatory pathologies are also of interest such as rheumatoid arthritis or chronic inflammatory bowel diseases (IBD), such as Crohn's disease or ulcerative colitis, secondary excessive inflammation caused by a primary infection, for example bacterial, parasitic but also viral, such as influenza, infant bronchiolitis, or SARS (Severe Acute Respiratory Syndrome).

Other features and advantages will emerge from the detailed description of the invention, examples and figures that follows.

### BRIEF DESCRIPTION OF THE FIGURES

FIG. 1 shows the dose-effect of budesonide on BEAS-2B cells. BEAS-2B cells were co-incubated with TNF-α (1 pg/mL) alone or with different doses of budesonide (1.5; 0.75; 0.5; 0.1 nM). After 6 hours of incubation, the IL-8 concentration was measured in the supernatant. IL-8 levels are expressed as a percentage of the IL-8 level induced by stimulation with TNF-α for 6 hours. The results correspond to the mean+SEM for at least 3 experiments, n=6-12 technical replicates. *p<0.05 and ***p<0.001 compared with the TNF-α control group. The “medium” condition corresponds to the negative control, showing that the cells do not produce IL-8 in the absence of TNF-α.

FIG. 2 shows the in vitro effect of the combination of E. faecalis and budesonide on BEAS-2B cells. BEAS-2B cells were co-incubated with TNF-α (1 pg/mL) alone or with a strain of E. faecalis (CNCM I-4969; I-5701; I-5699) MOI 10:1, and/or 0.1 nM of budesonide, or 0.5 nM of budesonide. After 6 hours of incubation, the IL-8 concentration was measured in the supernatant by ELISA. IL-8 levels are expressed as a percentage of the IL-8 level induced by stimulation with TNF-α for 6 hours. The results correspond to the median of 3 experiments, n=9 technical replicates. *p<0.05 and ***p<0.001 compared with the TNF-α control group, by a Kruskal-Wallis non-parametric test followed by Dunn's post-test.

FIG. 3 shows the ex vivo effect of the combination of E. faecalis (CNCM I-4969) and budesonide on mouse lung explants. Mouse lung explants were cultured alone with E. faecalis (CNCM I-4969) 50 CFU, and/or 0.1 nM of budesonide, or 0.5 nM of budesonide. After 24 hours of incubation, the concentration of different cytokines was measured in the supernatant by multiplex assay using Luminex technology. The cytokine levels are expressed as a percentage of the levels measured in the medium condition at 24 hours. The results correspond to the mean+SEM of an experiment, n=3 biological replicates per condition. *p<0.05, **p<0.01 and ***p<0.001 compared with the medium control.

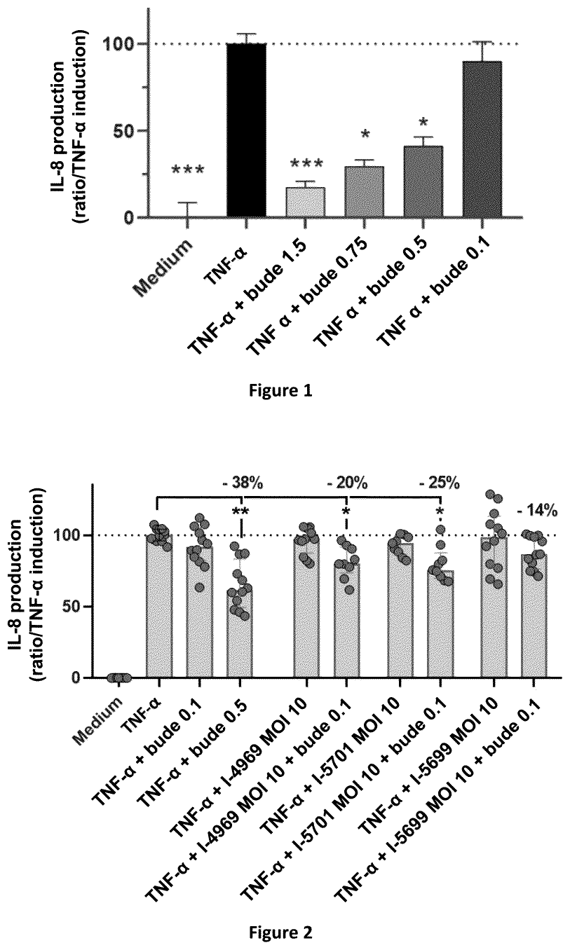

### DETAILED DESCRIPTION OF THE INVENTION

### Definition

The term “bacterium” in the sense of the invention is understood to mean a single-cell microorganism capable of being reproduced by cell division. The bacteria are classified by family, genus, species. Each bacterial species comprises a diversity of bacterial strains. The bacterial strain according to the invention belongs to the family Enterococcaceae, the genus Enterococcus and the species faecalis.

Therefore, “bacterial strain” or “strain” within the meaning of the invention is a specific bacterial strain but also all the bacteria derived from the strain or obtained from the strain or corresponding to the bacterial strain and having the same metabolic functions, for example at least one bacterium removed from a colony derived from the strain. Preferentially, a bacterial strain according to the invention is also understood to mean one of the strains deposited under number CNCM I-4969 on Apr. 14, 2015 at the National Collection of Cultures of Microorganisms (CNCM), 25 rue du Docteur Roux, 75724 Paris Cedex 15, France, but also under number CNCM I-5701 and CNCM 1-5699 deposited on Jun. 16, 2021 at the National Collection of Cultures of Microorganisms (CNCM), 25 rue du Docteur Roux, 75724 Paris Cedex 15, France as well as the derived strains.

The term “derived strain” within the meaning of the invention refers to a bacterial strain having a strong similarity with one of the bacterial strains deposited under number CNCM I-4969, or CNCM 1-5701 or CNCM I-5699. Preferentially, the derived strain comprises a nucleotide sequence having at least 99% ANI identity with the nucleotide sequence of the chromosome of at least one of strains CNCM I-4969, or CNCM I-5701 or CNCM I-5699, more preferentially, at least 99.91%, at least 99.92%, at least 99.93%, at least 99.94%, at least 99.95%, at least 99.96%, at least 99.97%, at least 99.98%, at least 99.99% ANI identity with the nucleotide sequence of the chromosome of one of the strains CNCM I-4969 or CNCM I-5701 or CNCM I-5699.

For the purposes of the invention, “ANI” refers to the average percentage identity of the nucleotides calculated from the pair-wise comparison of all the chromosome sequences of the two bacterial strains. According to the general knowledge well known to the person skilled in the art, the genomic DNA can be extracted from said strain of interest from a pure bacterial culture from said strain of interest, followed by DNA sequencing according to various well-known methods, for example Sanger, Roche 454, Illumina, Oxford Nanopore, then the genome is sequenced and assembled by biocomputing and the sequences obtained are analyzed. Finally, the chromosome sequences of interest are compared in pairs to calculate the ANI.

The term “supernatant” within the meaning of the invention is understood to mean the culture supernatant of the bacterial strain according to the invention optionally comprising cellular components of said strain and/or cell debris of said strain, and/or metabolites and/or molecules secreted by said strain.

The term “human” within the meaning of the invention is understood to mean a human patient. Said human patient may belong to any age group, that is the patient may be an infant, a baby, a child, an adolescent or an adult. It is known that children, babies, infants and elderly people are more susceptible to respiratory diseases, in particular asthma. Preferentially, humans are resistant and/or refractory to corticosteroid treatments, for example budesonide.

Within the meaning of the invention, the term “patient resistant and/or refractory to corticosteroid treatment” is understood to be a patient who is receiving a corticosteroid-based treatment, who does not respond to said treatment, that is, in whom a reversal of the progression of inflammation or a reversal or regression of the disease progression is not observed.

“Prevention” within the meaning of the invention is understood to mean the reduction to a lesser degree of the risk or the probability of occurrence of a given phenomenon, that is, in the context of the present invention of the inflammation, preferentially respiratory diseases, for example asthma.

Withing the meaning of the invention, “treatment” means reducing the progression of the disease, stabilizing, reversing or regressing, or even interrupting or inhibiting the progression of the inflammation, preferentially the respiratory diseases, for example asthma. In the context of the invention, these terms also apply to one or more symptoms of said diseases of the present invention.

Within the meaning of the invention, “respiratory disease” is understood herein to mean diseases of the breathing apparatus or causing respiratory disorders.

Within the meaning of the invention, the term “acceptable excipient” is intended to mean any compound making it possible to facilitate the formulation of the composition and not modifying the nature of the biological activity of the active ingredient. An acceptable excipient may be a solvent, buffer, saline solution, plasticizer, lubricant, dispersion medium, absorption retarding agent, flow agent, isotonic agent. Preferentially, it may be pharmaceutically acceptable excipients that are selected according to the pharmaceutical form and the desired mode of administration, among the usual excipients known to the person skilled in the art and suitable for human and/or veterinary use. The excipient will thus be chosen as a function of the administration route, for example suitable for oral, intravenous, intramuscular, topical administration etc.

“Simultaneous administration” is understood to mean both administration in the form of a single pharmaceutical formulation combining the two active ingredients, E. faecalis and an anti-inflammatory agent such as a corticosteroid, as well as the separate administration of the two distinct pharmaceutical formulations each containing one of the two active ingredients taken simultaneously or within a very short time span (at most about 1 hour).

### Combination According to the Invention

The present invention therefore consists of a novel product which combines two active ingredients, hereinafter referred to as the combination according to the invention, on the one hand, strains of E. faecalis and, on the other hand, an anti-inflammatory agent, in particular a corticosteroid, thus making it possible to potentiate the anti-inflammatory effect of the corticoid and to reduce the useful concentration thereof and thus reduce undesirable effects in patients.

The present invention therefore relates to a combination comprising at least one bacterial strain of the species Enterococcus faecalis (i) and at least one anti-inflammatory agent (ii) for use thereof as a medicament in humans or animals.

The inventors previously discovered and isolated bacterial strains of E. faecalis from mice lungs, human feces or cheese, in which the inventors identified anti-inflammatory properties.

E. faecalis is an enterococcus naturally present in the digestive tract of humans and animals. It is notably responsible for nosocomial infections and can cause bacteremia, endocarditis, as well as infections of the urinary tract, prostate or epididymis.

Due to the wide genetic diversity within the species E. faecalis. A multi-locus sequence typing (MLST) method, based on the analysis of the nucleotide sequence of 7 loci (˜3 kb over 3.5 Mb) of the core genome, has made it possible to define more than 1000 genome types (ST for sequence type) (Ruiz-Garbajosa 2006; Neumann 2019). The ST then having at least 6 alleles on the 7 loci are considered to belong to the same clonal complex (CC). Finally, next-generation sequencing has made it possible to develop a genome-wide sequence typing method (cgMLST for core-genome multi-locus sequence typing) which, for E. faecalis, uses 1972 loci, that is, more than half of the genome (Neumann 2019), which has allowed the previous data obtained with MLST genotyping to be confirmed.

In this case, said strains of the species E. faecalis preferentially belong to the genomic group CC40 or CC21.

According to another preferred object, said strain present in the combination according to the invention comprises a 16S rRNA sequence of SEQ ID NO: 1.

According to a particularly preferred embodiment, the strain is chosen from the strain deposited under number CNCM I-4969, CNCM I-5701 and CNCM 1-5699.

The strain CNCM I-5699 belongs to clonal complex 21, more particularly to the genomic type ST21 and has a chromosome of 2843733 bp (base pair). The strain CNCM I-5701 and the strain CNCM I-4969 both belong to clonal complex 40, respectively to the genomic type ST284 and ST40, and their chromosome respectively has 2982831 bp and 3056663 bp.

The bacterial strain or optionally the mixture of bacterial strains present in the combination according to the invention can be in any form producing the desired effects and efficacy described in the present application. In particular, the strain can be in living (cultivable or not), dead, semi-live, attenuated or inactivated form.

These forms can be obtained by various techniques well known to the person skilled in the art. Mention will be made, for example, of the following techniques: irradiation, thermal inactivation or freeze-drying, in particular by heat, exposure to an appropriate pH, UV, gamma rays, X-rays or high pressure.

The term “semi-living” refers to a bacterium with a low physiological activity whose ability to proliferate is reduced, temporarily or definitively. The term “inactivated” refers to a bacterium which is no longer capable, temporarily or definitively, of proliferating. The term “dead” refers to a bacterium that is definitively no longer capable of proliferating. The dead or inactivated bacteria can have intact or broken cell membranes. Thus, the term “inactivated” also refers to the obtained extracts and lysates of bacteria. It is particularly advantageous to use such forms in the pharmaceutical compositions of the invention.

According to one variant, the combination according to the invention comprises a bacterium of E. faecalis and/or the culture supernatant obtained from one of the bacterial strains of E. faecalis according to any of the embodiments described above.

When the combination comprises a supernatant, the latter can be obtained from one of the strains deposited under number CNCM I-4969, CNCM I-5701 and CNCM I-5699 or a mixture of several strains of E. faecalis, preferentially of a mixture of the strains deposited under number CNCM I-4969, CNCM I-5701 and CNCM I-5699.

Thus, the combination according to the invention comprises at least one bacterial strain and/or a culture supernatant according to one of the embodiments described above, and an anti-inflammatory agent.

The anti-inflammatory agent is preferentially a corticosteroid type agent. When the anti-inflammatory agent is a corticosteroid, it is preferentially budesonide.

Corticosteroids refer to both hormones synthesized by the adrenal glands and anti-inflammatory medicaments. Within the meaning of the present invention, the term relates to anti-inflammatory medicaments. These are synthetic hormones used in treatment for their anti-inflammatory properties. They may be effective in the short term, medium term and in the longer term in the treatment of inflammatory and/or auto-immune diseases, such as rheumatoid arthritis, systemic lupus erythematosus and certain respiratory disorders.

Preferentially, the combination of E. faecalis and the anti-inflammatory agent is intended to be administered simultaneously or sequentially over time as a medicament.

### Composition According to the Invention

The combination disclosed hereinbefore is advantageously administered in a composition, preferentially a pharmaceutical composition.

Thus, the invention relates to a composition comprising the combination according to the invention and at least one acceptable excipient, preferentially a pharmaceutically acceptable excipient.

The composition may optionally comprise a physiologically acceptable medium, that is, a medium that is compatible with the microorganism but also the organism of the individual to which said composition is to be administered. It may involve, for example, a non-toxic solvent such as water, buffer, saline solutions. In particular, said medium is compatible with oral administration.

Thus, the composition comprises at least one bacterial strain of the species E. faecalis, preferentially selected from the strain deposited under number CNCM I-4969, CNCM I-5701, CNCM I-5699 and a mixture thereof; and/or the culture supernatant obtained from said strains and at least one acceptable excipient and/or physiologically acceptable medium.

Optionally, the composition according to the invention may comprise in addition to the combination according to the invention, at least one additional compound. The additional compound may be an ingredient, a molecule, a third active ingredient, a microorganism, a bacterium or a mixture of bacteria.

The composition according to the invention is in any form acceptable to be administered to a patient, preferentially a human or animal (non-human) patient. Thus, the composition according to the invention also relates to veterinary uses.

The composition may be in any suitable form. Thus, the composition according to the invention may be in a form chosen from a powder, powder to be nebulized, microencapsulated powder, microencapsulated powder to be nebulized, gelatin capsule, capsule, tablet, lozenge, granules, solution, solution to be nebulized, emulsion, emulsion to be nebulized, suspension, suspension to be nebulized, ampoule, suppository, inhaler and syrup, more preferentially in the form of an inhaler such as a nebulizer.

Preferably, the composition according to the invention is in solid, liquid or freeze-dried form, more preferentially a liquid capable of being nebulized or solid in powder form.

When the composition is in liquid form, it comprises the anti-inflammatory agent and at least one bacterial strain according to the invention and/or derived strain and/or a culture medium physiological acceptable for said bacteria, which makes it possible to maintain them, such as for example the BHI medium, or an equivalent medium not containing any derived product of animal origin.

When the composition according to the invention is in solid form, the anti-inflammatory agent and the bacterial strains according to the invention and/or the derived strain can be present in freeze-dried form, and may also comprise excipients such as, for example, microcrystalline cellulose, lactose, sucrose, fructose, levulose, starches, stachyose, raffinose, amylum, calcium lactate, magnesium sulfate, sodium citrate, calcium stearate, polyvinylpyrrolidone, maltodextrin, galactooligosaccharides, fructooligosaccharides, pectins, beta-glucans, lactoglobulins, isomaltooligosaccharides, polydextroses, mannitol, sorbitol, glycerol, silicon dioxide, magnesium stearate, cysteine, mannose, galactose, anhydrous glucose, glucose monohydrate and/or mixtures thereof.

The person skilled in the art will know to choose the best routes and modes of administration of the composition according to the invention, as well as optimal posologies and dosage forms, according to the criteria generally taken into account in the manufacture of a medicament or the establishment of a pharmaceutical or veterinary treatment. Preferably, the composition according to the invention is in a form suitable for oral, nasal, parenteral, rectal, sublingual, ocular, atrial, intramuscular, intravenous, inhalation or cutaneous administration, more preferentially by nasal or inhalation route, using a nebulization apparatus.

The composition may comprise 0.05 mg to 4 mg per day, preferentially 0.25 mg to 4 mg per day. When the patient is an adult, the composition may comprise 0.1 mg to 4 mg per day, preferentially 0.5 mg to 4 mg per day. When the patient is a child, the composition may comprise 0.05 mg to 2 mg per day, preferentially 0.25 mg to 2 mg per day.

In a particularly preferred manner, the composition comprises at least 10³ colony forming units (CFU) of bacteria per daily dose of composition to be administered, preferentially 10⁴ to 10⁹ CFU, more preferentially 10⁵ to 10⁷ CFU.

Preferentially, this corresponds to a daily dose of bacteria to be administered, regardless of the weight of the person or of the animal. Thus preferably, this dose is administered as a single dose. The useful composition then comprises at least 10³, preferentially 10⁴ to 10⁹ CFU of bacteria per daily dose to be administered, even more preferentially 10⁵ to 10⁷ CFU.

Said bacteria are a mixture of bacteria corresponding to or derived from one of the strains according to the invention, including the derived strains.

The term CFU (Colony Forming Unit) is a unit making it possible to count the number of bacteria capable of giving rise to a colony during propagation, that is to say viable bacteria. It should be understood that non-viable bacteria can also be present in the compositions and that in general they should not have a negative effect on the properties of the living bacteria of the composition.

Preferentially, the daily dose is measured per gram or milliliter of the final composition according to the invention.

According to another preferred object, when the composition according to the invention comprises a mixture of living bacteria or not, said mixture comprising at least living bacteria, the composition preferentially comprises at least 1% of living bacteria (in number), more preferentially at least 10% living bacteria (in number), even more preferentially at least 50% living bacteria (in number).

The living bacteria are a mixture of bacteria corresponding to one of the strains according to the invention, including derived strains, preferentially belonging to clonal complex CC40 or CC21, more preferentially a strain having a 16S RNA sequence of SEQ ID NO:1.

In the case of the implementation of a supernatant of any one of the aforementioned bacterial strains, the composition according to the invention including said supernatant may in particular comprise a content thereof of between 0.1 and 99% by weight, namely from 5 to 95% by weight, in particular from 10 to 90% by weight and more particularly from 15 to 85% by weight, relative to the total weight of the composition.

Thus, a composition according to the invention may comprise a supernatant content of between 0.1 and 99% by weight, namely from 5 to 95%, in particular from 10 to 90% by weight, more particularly from 15 to 85% by weight, relative to the total weight of the composition.

When the composition is intended for therapeutic use, the composition comprises at least one pharmaceutically acceptable excipient, the composition according to the invention is then a pharmaceutical composition.

The term “pharmaceutically acceptable excipient” herein means an excipient that is not accompanied by significant harmful effects when administered to an individual. Pharmaceutically acceptable excipients are well known to the person skilled in the art.

Thus, the invention also relates to a pharmaceutical composition comprising the ingredient(s) included in the composition according to the invention and according to one of any embodiments described above. Said pharmaceutical composition may be a medicament.

According to one variant, the invention relates to a pharmaceutical kit comprising at least one strain E. faecalis, preferentially the strain CNCM I-4969 or CNCM 1-5701 or CNCM I-5699 or mixtures thereof and a corticosteroid preferentially budesonide, for simultaneous or sequential administration over time.

In the pharmaceutical kit according to the invention, the strain E. faecalis and the corticosteroid such as budesonide may be present in the form of a single pharmaceutical formulation combining the two active ingredients 1) E. faecalis and 2) the corticosteroid, or else two distinct formulations each comprising one of the active ingredients (then allowing simultaneous or sequential administration).

### Use

The composition or the kit according to the invention is useful as a medicament for human or veterinary health use.

Preferentially, the composition according to the invention is intended to prevent and/or treat inflammation and thus be used in the treatment and/or prevention of inflammatory diseases, more preferentially respiratory diseases and/or inflammatory diseases of the intestine and/or rheumatoid arthritis and/or inflammatory diseases associated with a viral, parasitic or bacterial infection.

By way of example, mention may thus be made of asthma (mild, moderate or severe), for example, bronchial, allergic, intrinsic, extrinsic, exercise-induced, allergen-induced, such as dust, steroid-resistant asthma; bronchitis, including infectious and eosinophilic bronchitis, chronic obstructive pulmonary disease (COPD), such as chronic obstructive pulmonary disease (COPD), cystic fibrosis, pulmonary fibrosis, including cryptogenic fibrosing alveolitis, idiopathic pulmonary fibrosis, idiopathic interstitial lung disease, fibrosis complicating antineoplastic therapy and chronic infection, including tuberculosis and aspergillosis and other fungal infections; vasculitis and thrombotic events of the pulmonary vascular system and pulmonary arterial hypertension; antitussive activity comprising treatment for chronic cough associated with inflammatory and secretory disorders of the respiratory tract and iatrogenic cough; acute and chronic rhinitis, including drug-induced rhinitis, and vasomotor rhinitis; perennial and seasonal allergic rhinitis including nervous rhinitis (hay fever); nasal polyps; acute viral infection, including cold, and infection due to respiratory syncytial virus (RSV), influenza, pulmonary lung disease, coronavirus infections (particularly SARS) and adenovirus, pulmonary edema, pulmonary embolism, pneumonia, pulmonary sarcoidosis, silicosis, farmer's lung and related diseases, hypersensitivity pneumonitis, respiratory failure, acute respiratory distress syndrome, emphysema, chronic bronchitis, tuberculosis and lung cancer.

The composition according to the invention also relates to respiratory pathologies having common “immune disorders” with those observed in asthma such as the induction of certain cytokines (IL-6, TSLP, IL-8, IL-5, IL-13, IL-17) and mucosal disorders (hyperproduction of mucus) and modifications of the bronchial epithelium.

In particular, uses of the composition according to the present invention encompass the prevention and treatment of chronic respiratory diseases, for example asthma, COPD and rhinitis. Even more particularly, uses of the composition according to the present invention relate to the prevention and treatment of asthma. The use of the strain of the invention and the composition comprising it can therefore notably be used effectively to prevent the onset of asthma in patients known for their predisposition to this pathology (for example, patients who have a family predisposition to the development of asthma).

Given the common inflammatory pathways, for example the production of interleukin such as IL-8, the composition according to the invention is also useful in a disease selected from Crohn's disease, ulcerative colitis, diverticulitis, esophagitis, gastritis, pancreatitis, gastro-duodenal ulcer and irritable bowel syndrome.

Finally, the composition according to the invention is of particular interest when humans or animals are resistant and/or refractory to corticosteroids.

### Method for Preparing a Composition According to the Invention

The combination or composition according to the invention can be produced by any means well known to the person skilled in the art.

By way of example, the strain according to the invention is produced by culture, for example, in a growth medium known to the person skilled in the art (for example, a BHI: “Brain-Heart Infusion” medium) for 8 hours to 3 days, at a temperature of 30-37° C., with or without pH adjustment. The fermentation broth containing the bacterial cells is collected. The broth can be used as is, concentrated or freeze-dried. Advantageously, the bacteria will be collected, for example by centrifugation and then resuspended in an appropriate buffer, for example PBS (“phosphate buffered saline”). The bacterial concentration can be established using flow cytometry or another equivalent method.

The bacteria can then be nebulized from the liquid form or freeze-dried form. A solvent may be added in order to facilitate nebulization.

The invention is now shown by non-limiting examples of composition according to the invention and by results.

### EXAMPLES

### Example 1—Minimum Dose of Budesonide Alone on BEAS-2B Cells

Budesonide is a corticosteroid with anti-inflammatory properties in vitro and in vivo and used in humans, particularly in the treatment of asthma.

Human bronchial cells (BEAS-2B) are cultured and a pro-inflammatory situation is induced via the addition of TNF-α to the medium. The BEAS-2B cells then respond by releasing IL-8 into the medium. The IL-8 assay after 6 hours is used to estimate the activation state of the cells, which can be reduced by adding an anti-inflammatory agent.

The inventors thus identified a dose of budesonide which induces little or no anti-inflammatory effect in the BEAS-2B model, that is, 0.1 nM.

### Example 2—Anti-Inflammatory Effects In Vitro of the Combination According to the Invention

The inventors then sought to test the effects of the combination according to the invention, that is E. faecalis and budesonide on the BEAS-2B model.

For this, they used 3 distinct strains: CNCM I-4969; CNCM I-5701 and CNCM I-5699. According to the genome analyses, these three strains are phylogenetically very close since the sequence homology of the 16S RNA gene of said three strains is 100%. Despite a sequence homology of the 16S RNA, the three strains notably have different anti-inflammatory properties, in particular a different cytokine profile. This results in a different efficacy for each strain.

The strains are initially added alone. The 3 strains CNCM I-4969, CNCM I-5701 and CNCM I-5699 with a MOI 10:1, do not have any effect on the release of IL-8 induced by TNF-α (FIG. 2), like the 0.1 nM dose of budesonide.

Secondly, during the co-incubation of the BEAS-2B cells stimulated with TNF-α with 0.1 nM of budesonide and one of the strains of E. faecalis (CNCM 1-4969 or CNCM 1-5701 or CNCM I-5699), the inventors observed a synergistic effect of the combination according to the invention which induces the inhibition of IL-8 release by 32%, 31% and 12% respectively with strains CNCM I-4969, CNCM I-5701 and CNCM I-5699.

The strain of E. faecalis CNCM I-5699, combined with 0.1 nM of budesonide tends to reduce IL-8 induced by TNF-α by 12%.

The effect of the combination of budesonide 0.1 nM and CNCM I-4969 or CNCM I-5701 causes an inhibition of 30%, whereas budesonide alone and CNCM I-4969 or CNCM 1-5701 alone have no effect, thus demonstrating the synergistic action of the combination according to the invention compared to budesonide or E. faecalis administered alone (FIG. 2). Inhibition with a 5-times higher dose of budesonide alone is 50%.

### Example 3—Test of Anti-Inflammatory Effects Ex Vivo of the Combination According to the Invention

The inventors continued their work on the potentiating effect of E. faecalis on a more complex model of lung explants of axenic mice in ex vivo culture. These explants have a basal production of different cytokines, which can be modified by the presence of immunomodulators.

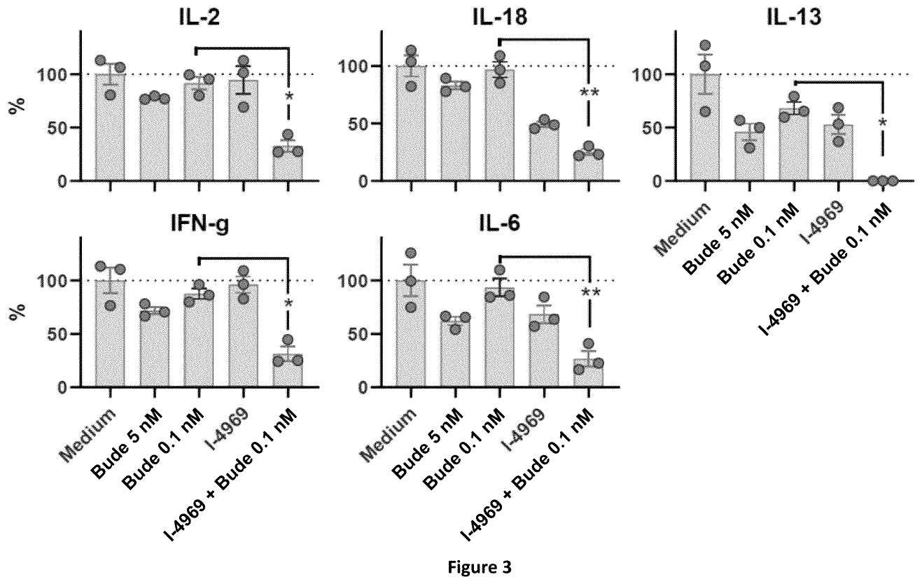

The inventors then observed that 0.1 nM of budesonide and 50 CFU of CNCM I-4969 alone had no effects, or modest effects on the cytokines measured in the explant supernatant (FIG. 3).

However, during co-incubation with a combination of 0.1 nM of budesonide and 50 CFU of E. faecalis and CNCM I-4969, the inventors observed a synergistic effect inducing a strong inhibition of the release of pro-inflammatory cytokines IL-2, IL-18, IL-13, IFN-r and IL-6.

The combination according to the invention (budesonide 0.1 nM-CNCM I-4969) makes it possible to obtain an anti-inflammatory effect greater than that obtained with a dose of budesonide alone that is 50 times higher, here again demonstrating the synergistic effect of the combination according to the invention (FIG. 3).

In conclusion, the inventors were able to demonstrate that the combination associating the strains of E. faecalis and budesonide obtained a synergistic anti-inflammatory effect. Thus, a dose of corticosteroid which alone has no anti-inflammatory effect, shows significant efficacy in vitro and ex vivo when combined with a strain of E. faecalis.

1. A combination of at least one bacterial strain of the species Enterococcus faecalis (i) and an anti-inflammatory agent (ii) for use as a medicament in humans or animals.

2. The combination according to claim 1, characterized in that the anti-inflammatory agent is a corticosteroid.

3. The combination according to claim 1, characterized in that the strain comprises a sequence of the 16S rRNA gene having at least 99% similarity with the sequence SEQ ID NO:1.

4. The combination according to claim 1, characterized in that the strain is chosen from the strain deposited under number CNCM I-4969, CNCM I-5701 and CNCM I-5699 and/or the corticosteroid is budesonide.

5. The combination according to claim 1, characterized in that the strain is living, dead, attenuated or inactivated.

6. A pharmaceutical composition comprising the combination according to claim 1 and at least one pharmaceutically acceptable excipient.

7. The pharmaceutical composition according to claim 6, characterized in that the living bacteria represent at least 10³ CFU.

8. The composition according to claim 6, characterized in that it is in solid, liquid or freeze-dried form.

9. The composition according to claim 6, for use in the prevention and/or treatment of respiratory diseases and/or inflammatory diseases of the intestine and/or rheumatoid arthritis and/or inflammatory diseases associated with a viral, parasitic or bacterial infection.

10. The composition according to claim 9, for use in the prevention and/or treatment of a disease selected from asthma, bronchitis, bronchiolitis, COPD, pulmonary fibrosis, or rhinitis.

11. The composition according to claim 6, characterized in that the human or animal is resistant to corticosteroids.


SEQUENCE LISTING (1 sequence)

SEQ ID NO: 1 - DNA, 1522 bp, Enterococcus faecalis
  source 1..1522: mol_type = other DNA
agagtttgatcctggctcaggacgaacgctggcggcgtgcctaatacatgcaagtcgaacgcttctttcctcccgagtgcttgcactcaattggaaagaggagtggcggacgggtgagtaacacgtgggtaacctacccatcagagggggataacacttggaaacaggtgctaataccgcataacagtttatgccgcatggcataagagtgaaaggcgctttcgggtgtcgctgatggatggacccgcggtgcattagctagttggtgaggtaacggctcaccaaggccacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggacgttagtaactgaacgtcccctgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgctagaccgcgaggtcatgcaaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgattggggtgaagtcgtaacaaggtagcc